(19) United States
(12) Patent Application Publication
(10) Pub. No.: US 2004/0225000 A1
(43) Pub. Date: Nov. 11, 2004

(54) Methods of producing 4-amino-3-mercapto-triazoles

(76) Inventors: Ned H. Heindel, Easton, PA (US); Jeffrey D. Laskin, Piscataway, NJ (US); Diane E. Heck, Rumson, NJ (US); Christophe Guillon, Bethlehem, PA (US); Robert D. Rapp, Reading, PA (US)

Correspondence Address: PERKINS COIE LLP, POST OFFICE BOX 1208, SEATTLE, WA, 98111-1208, US

(21) Appl. No.: 10/863,785

(22) Filed: Jun. 8, 2004

Related U.S. Application Data
(63) Continuation of application No. 10/251,151, filed on Sep. 19, 2002, now abandoned.
Application No. 10/251,151 is a continuation-in-part of application No. 09/790,330, filed on Feb. 21, 2001, now abandoned.

(30) Foreign Application Priority Data
Aug. 21, 1999 (WO) PCT/US99/19146

(51) Int. Cl. C07D 249/14; A61K 31/4196

(52) U.S. Cl. 514/383; 548/263.8

(57) ABSTRACT

This invention claims the process for producing two structural variants of functionalized 4-amino-3-mercapto-1,2,4-triazoles as inhibitors of nitric oxide synthase (NOS) and as inhibitors of malignant cell growth. This fundamental molecular construct operates as a heterocyclic mimic of the open-chain N-aminoarginines (or N-aminoguanidines) previously established as NOS inhibitors.

### CROSS-REFERENCE TO RELATED APPLICATIONS

This application is a continuation-in-part of U.S. patent application Ser. No. 09/790,330, filed Feb. 21, 2001, which is the U.S. national phase of International Application No. PCT/US99/19146, filed Aug. 21, 1999, which claims priority to U.S. Provisional Patent Application No. 60/242,160, filed Aug. 21, 1998, the disclosures of which are incorporated by reference herein in their entirety.

### BACKGROUND OF THE INVENTION

From a clinical perspective it is very clear that in vivo pharmacological manipulation of nitric oxide (NO) production will be of considerable therapeutic value. The list of nitric oxide synthase mediated diseases grows longer every year but the broad classes of dysfunctions includes many gastrointestinal motility problems, inflammatory states, and neurodegenerative disorders. A partial array of specific medical circumstances which appear to be certainly associated with NOS are: sunburn, rheumatoid arthritis, ulcerative colitis, Crohn's disease, lupus, septic and toxic shock, asthma, hypertension, myocarditis, diabetes, and may autoimmune and respiratory problems (Macdonald-1996).

Now that it is known that the various isoforms of NOS utilize the arginine to citrulline deamination as the route to NO, many therapeutic drugs have been designed to target that pathway (Kerwin-1994). With a wide variety of N-gamma-substituted arginines identified as inhibitors of NOS bearing such pendant gamma residues as nitro, amino, and even alkyl, and with the observation that some heterocyclic triazole systems appear to mimic the guanidino portion of arginine (Buchmuller-Rouiller-1992), we proposed the use of planar, fused-ring bio-isosteric models of arginine as new candidate classes of NOS inhibitors.

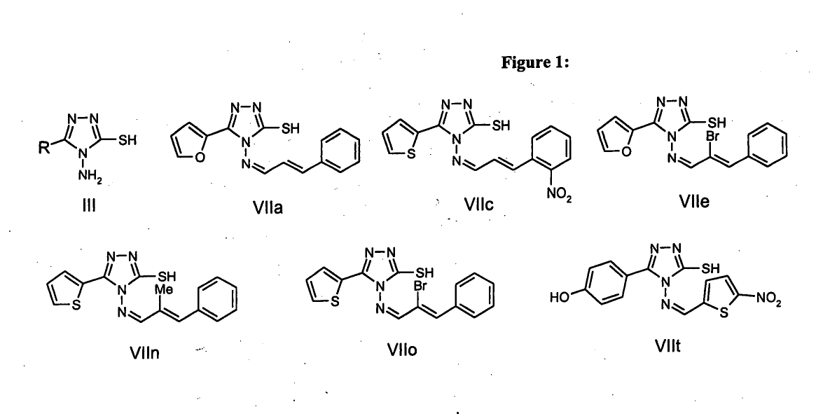

While 1,2,4-triazoles do have an abundant patent literature as useful agriculturals and even as human therapeutics (Camden-1 998, Tomioka-1998, and Reitz-1997), the specific prior art on the 4-amino-1,2,4-triazoles indicated they did not possess inhibitory activity against nitric oxide synthase (NOS) (Buchmuller-Rouiller-1992). We have found, however, significant NOS-inhibitory activity in that 4-amino-1,2,4-triazole family bearing a pendant 3-mercapto moiety. Furthermore, active NOS inhibitors were also found and in several of the N- or S-functionalized derivatives of these 4-amino-3-mercapto-(4H)-1,2,4-triazoles, see FIG. 1.

We believe these heterocyclic candidate therapeutics are functioning as cyclic biological isosteres of the N-aminoguanidines previously shown to possess NOS inhibition (Macdonald-1996).

We also report herein the utility of Classes VII and X as anticancer therapeutics active against a broad array of malignant cell types, a pharmacology without precedent in these families. Camden has reported that certain specially substituted N-alkyl-1,2,4-triazoles do display antineoplastic activity (Camden-1998). None of our anticancer families is N-alkyl substituted.

### DETAILED DESCRIPTION OF THE INVENTION

The present invention lies in the synthesis of two novel structural variants: 4-amino-3-mercapto-triazoles; 4-(R′-Imino)-3-Mercapto-5-(R)4H-1,2,4-Triazole (VII) and 4-(R′-imino)-3-alkylthio-5-R-1,2,4-triazoles (X). This fundamental molecular construct-operates as a heterocyclic mimic of the open-chain N-aminoarginines (or N-aminoguandines) previously established as NOS inhibitors. In addition, novel process are described to obtain diverse members of these 4-amino-3-mercapto-1,2,4-triazoles.

In each of the structures depicted herein, R and R′, which may be the same or different, are alkyl, aryl, hydrogen, fluoroalkyl, or heterocyclic moieties. By alkyl is meant any monovalent radical having the structure C.sub.nH.sub.2n+1-, especially lower alkyl radicals of between 1 and 6 carbons in length; by aryl is meant any organic radical derived from an aromatic hydrocarbon by the removal of one atom, for example phenyl or substituted phenyl radicals; by haloalkyl is meant a alkyl radical, especially a lower alkyl radical which carries a halide moiety as for example a fluoroalkyl, bromoalkyl, or chloroalkyl; and by heterocyclic is meant a cyclic ring structure, especially a heterocyclic structure having from 5 to 8 atoms in the ring. Especially, among the radicals included in the broad definition of these moieties are hydrogen, bromine, chlorine, methyl, cyclohexyl, phenyl, 2-thienyl, 2-furyl, 3-pyridyl, 2-phenylethyl, trifluoromethyl, C₆H₅-,p-F—C₆H₄—, 4-F—C₆H₄—, 2-Br—C₆H₄—, o-hydroxyphenyl, 2,3-dihydrophenyl, β-Me-butyrate, β-phenyl-butyrate, β-phenylpropionate methyl ester, 4-hydroxy-2-butyl, 4-chloro-2-butyl, Ph—CH₃CH₂—, cinnamaldehyde, —CH₂CH₂COOMe,—CH(CH₃)CH₂CH₂C-1, CH(CH₃)CH₂CO₂(C₆H₅), —CH═CH—Ph, —CH═CH-2-methoxyphenyl,—CH═CH-2-nitrophenyl, —CH═CH-(o-methoxyphenyl), α-bromocinnamaldehyde, —CH═CH-(o-nitrophenyl), α-chlorocinnamaldehyde, and α-methylcinnamaldehyde. More particularly, R may be selected from the group of methyl, cyclohexyl, phenyl, 2-thienyl, 2-furyl, 3-pyridyl, 2-phenylethyl, C₆H₅—,p-F—C₆H₄—, 4-F—C₆H₄—, o-hydroxyphenyl, Ph-CH₂CH₂—, CH═CH-Ph, -nitrophenyl, and 2-Br—C₆H₄—; and R′ may be selected from the group of hydrogen, bromine, chlorine, phenyl, 2-phenylethyl, C₆H₅—, p-F—C₆H₄—, 4-F—C₆H₄—, 2-Br—C₆H₄—, o-hydroxyphenyl, Ph-CH₂CH₂—, cinnamaldehyde,—CH₂CH₂COOMe, —CH(CH₃)CH₂CH₂Cl, —CH(CH₃)CH₂CO₂(C₆H₅), —CH═CH-Ph, —CH═CH-2-methoxyphenyl, —CH═CH-2-nitrophenyl, —CH═CH-(o-methoxyphenyl), α-bromocinnamaldehyde, —CH═CH-(o-nitrophenyl), α-chlorocinnamaldehyde, and α-methylcinnamaldehyde. In addition to these specified radicals, others may appear within the following examples.

More specifically, the present invention describes a syntheses generating unique N- and S-functionalized derivatives of these 4-amino-3-mercapto-4H-1,2,4-triazoles, viz the 4-(R′-imino)-3-mercapto-5-R-1,2,4-triazoles (general formula VII) and the 4-(R′-imino)-3-alkylthio-5-R-1,2,4-triazoles (general formula X):preparation of 4-(R′-imino)-3-mercapto-5-(R)-4H-1,2,4-triazole.

Triazoles of General Formula VII

Specifically, 4-imino-(cinnamyl)-3-mercapto-5-(2-thienyl)-4H-1,2,4-triazole (compound VIIa) was prepared by the condensation of 4-imino-3-mercapto-5-(2-thienyl)-4H-1,2,4-triazole, 100 mg (0.0504 mmol) with cinnamaldehyde, 123 mg (0.756 mmol) in 2 ml of absolute ethanol. The reaction was refluxed overnight, after which the product precipitated out of solution. The product was isolated via suction filtration and washed with cold ethanol to give 100 mg of product.

Similarly, by the same reaction a derivative of the 4-amino-3-mercapto-5-(2-thienyl)1,2,4-triazole (compound VIIb, i.e., the o-methoxycinnamyl derivative) was obtained; and from o-nitrocinnamaldehyde and the same 4-amino-3-mercapto-5-(2-thienyl)1,2,4-triazole compound VIIc was obtained.

This reaction is, in fact, general for any 4-amino-3-mercapto-5-(R)-1,2,4-triazole, i.e., any compound defined by general formula III wherein R is methyl, cyclohexyl, phenyl, 4-fluorophenyl, 2-pyridyl, 3-pyridyl, 4-pyridyl, 2- or 3-furyl, or any other aryl, heterocyclic or alkyl moiety, in chemical condensation with any substituted cinnamaldehyde. Some specific examples (with R defined as shown and the substituted cinnamaldehyde component listed by name) are included in Table 1:

| Compound | R | Cinnamaldehyde compound | Yield (%) | mp (° C.) |
|---|---|---|---|---|
| VIIa | 2-thienyl | cinnamaldehyde | 64 | 206-206.5 |
| VIIb | 2-thienyl | o-methoxycinnamaldehyde | 75 | 227-228 |
| VIIc | 2-thienyl | o-nitrocinnamaldehyde | 83 | 241-215 |
| VIId | 2-furyl | cinnamaldehyde | 84 | 181-182 |
| VIIe | 2-furyl | α-bromocinnamaldehyde | 84 | 184-185 |
| VIIf | Methyl | cinnamaldehyde | 59 | 193-194 |
| VIIg | Methyl | α-bromocinnamaldehyde | 54 | 194-195 |
| VIIh | Methyl | α-chlorocinnamaldehyde | 88 | 221.5-222.5 |
| VIIi | Cyclohexyl | α-bromocinnamaldehyde | 97 | 199-200 |
| VIIj | Ph-(CH₂)₂— | α-bromocinnamaldehyde | 67 | 175-176 |
| VIIk | Phenyl | α-bromocinnamaldehyde | 89 | 195-196 |
| VIIl | 4-fluorophenyl | cinnamaldehyde | 62 | 216-217 |
| VIIm | 4-fluorophenyl | α-bromocinnamaldehyde | 51 | 212-213 |
| VIIn | 2-thienyl | α-methylcinnamaldehyde | 45 | 200-201 |
| VIIo | 2-thienyl | α-bromocinnamaldehyde | 88 | 197-198 |
| VIIp | 2-thienyl | α-chlorocinnamaldehyde | 65 | 222-223 |

By way of the same experimental method shown above, employing a 1.0 to 1.5 ratio of any requisite member of the class of compounds defined by general formula III as shown in FIG. 1 to any aromatic or heterocyclic aldehyde in sufficient anhydrous ethanol to achieve solubility, one can obtain 40 to 65% yields of purified members of chemical class VII (wherein R′ is an aromatic or heteroaromatic moiety). Addition of well-dried molecular sieves as water-absorbents increases the field and facilitates the reaction. Specifically, a mixture of 100 mg (0.52 mmol) of general formula III (specifically wherein R was 4-hydroxyphenyl) and 123 mg (0.78 mmol) of 5-nitro-2-thiophene carboxaldehyde in 10 ml of ethanol containing 40 mg of molecular sieves was refluxed for 72 hours, filtered hot, evaporated to about 5 ml, and chilled to obtain 89 mg of compound VIIt. Compound VIIt had the following additional properties: 1H NMR (CD₃COCD₃) δ (ppm): 7.01 (d, J=8.9 Hz, Hα′); 7.80-7.85 (m, 3Hβ/α′); 8.11 (d, J=3.8 Hz, Hβ′); 8.99 (s, —OH); 10.72 (s, —N═CH—).

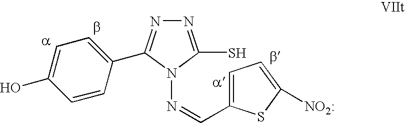

All imine members of class VII generated from aromatic and heterocyclic aldehydes in this fashion display a characteristic proton resonance for-N.dbd.CH— at 10.6+−0.3 ppm. Additional examples of such non-cinnamyl imines (VIIq-s) are shown in Table 2:

| | | Aromatic or | |
|---|---|---|---|
| Compound | R | heterocyclic aldehyde | Yield (%) |
| VIIq | 3-pyridyl | 5-nitro-2-thiophene | 64 |
| | | carboxaldehyde | |
| VIIr | 3-pyridyl | 2,3-dihydroxybenzaldehyde | 48 |
| VIIs | 3-pyridyl | 4-chlorobenzaldehyde | 57 |
| VIIt | 4-hydroxyphenyl | 5-nitro-2-thiophene | 49 |
| | | carboxaldehyde | |

Alkylation of the above indicated N-cinnamyl derivatives of those compounds defined by the structure of general formula VII possessing a ‘free’ thiol with alkylating species such as methyl iodide, dimethyl sulfate, ethyl iodide, and benzyl tosylate give the S-alkyl analogs defined by general formula X. This reaction and separation are accomplished in the following fashion.

Specifically, 139 mg (0.036 mmol) of 4-imino-(α-bromocinnamyl)-3-mercapto-5-cyclohexyl-4H-1,2,4-triazole compound VIIi, prepared in accordance with the process set forth above was dissolved in 5 ml of dry acetone and reacted with 60 μL (0.096 mmol) methyl iodide in the presence of 133 mg (0.096 mmol) of potassium carbonate for 3 hours at room temperature, the reaction flask being protected from light. The potassium carbonate was removed by filtration and the organic residue was filtered through a short column of flash silica gel.

Elution with methylene chloride 96%/Methanol 4% afforded 140.5 mg (97%) of a compound designated as Xi. Compound Xi had the following properties. ¹H NMR (CD₃COCD₃) δ (ppm): 1.26-1.44 (m, 3H); 1.60-1.72 (m, 3H); 1.78-1.87 (m, 2H); 1.95-2.02 (m, 2H); 2.66 (s, CH₃); 2.93 (tt, J=13.2 Hz, J′=3.5 Hz, Ha′); 7.49-7.55 (m, 3Hβ/γ), 8.06 (s, Hc); 7.99-8.05 (m, 2Hα); 8.66 (s, Ha).

| | |
|---|---|
| Anal. Calcd. For C₁₈H₂₁BrN₄S: | C,53.34%; H,5.22%; N,13.82% |
| Found: | C,53.19%; H,5.31%; N13.69% |

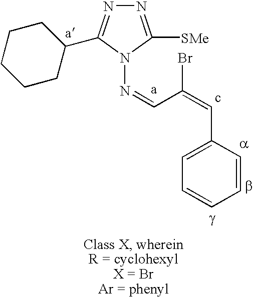

The ¹H NMR of Xi shows one characteristic singlet for Ha. Ha undergoes an upfield shift of almost 2 ppm (from 10.46 ppm in the starting compound VIII to 8.66 in Xi).

Similarly, utilizing this reaction a derivative of the 4-amino-5-[2-(phenyl)ethyl]-triazole, i.e., compound Xj was obtained in a 96% yield.

Furthermore, from VIIe was obtained Xe (R=2-furyl, X=Br and Ar=phenyl) in 96% yield from acetone.

This reaction is general for any 4-(R′-imino)-3-mercapto-5-(R)-4H-1,2,4-triazole, i.e., any compound defined by general formula VII where R is methyl, cyclohexyl, phenyl, 4-fluorophenyl, 2-pyridyl, 3-pyridyl, 4-pyridyl, 2-furyl, 3-furyl, or any other aryl, heterocyclic, or alkyl function, and R′ is the attachment arising from a condensation of the N—NH₂ moiety with any substituted cinnamaldehyde.

Properties of other members of class X prepared in accordance with this example are contained in Table 3:

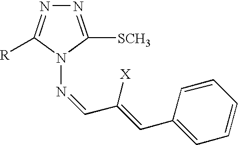

General structure for compounds of class X:

| Compound | R | X | Yield (%) | mp (° C.) |
|---|---|---|---|---|
| Xa | 2-thienyl | H | 75 | 250-252 |
| Xd | 2-furyl | H | 89 | 128-129.5 |
| Xe | 2-furyl | Br | 96 | 169-170 |
| Xf | Methyl | H | 63 | 125-126 |
| Xg | Methyl | Br | 65 | 100-102 |
| Xh | Methyl | Cl | 92 | 78.5-80 |
| Xi | Cyclohexyl | Br | 97 | 83.5-84.5 |
| Xj | Ph-(CH₂)₂— | Br | 96 | 115.5-116.5 |
| Xk | Phenyl | Br | 95 | 195-196 |
| Xo | 2-thienyl | Br | 80 | 123-124 |
| Xp | 2-thienyl | Cl | 62 | 130-131 |

Glossary

Pharmacologically Acceptable Composition: An amount of the relevant compound used for treatment of a patient.

Mammal: Refers to any animal classified as a mammal, including humans, domestic and farm animals, and zoo, sports and pet companion animals such as a household pet and other domesticated animal such as, but not limited to, cattle, sheep, ferrets, swine, horses, poultry, rabbits, goats, dogs, cats, and the like. Preferred companion animals are dogs and cats. Preferably, the mammal is human.

Patient: a mammal, preferably a human, in need of treatment of a condition, disorder or disease.

Treat and Treatment: Refer to both therapeutic treatment and prophylactic or preventative measures, wherein the object is to prevent or slow down (lessen) an undesired physiological condition, disorder or disease or obtain beneficial or desired clinical results. For purposes of this invention, beneficial or desired clinical results include, but are not limited to, alleviation of symptoms; diminishment of extent of condition, disorder or disease; stabilized (i.e. not worsening) state of condition, disorder or disease; delay or slowing of condition, disorder, or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Inhibitor: includes but is not limited to, any suitable molecule, compound, protein or fragment thereof, nucleic acid, formulation or substance that can regulate NOS activity in such a way that NOS is decreased. The inhibitor can include, but is not limited to the specifically identified Compounds VII and X.

Alkyl: refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms.

Aryl: Refers to and which is included with the term “carbocyclic ring structure” refers to an unsubstituted or substituted aromatic ring, substituted with one, two or three substituents selected from lower alkoxy, lower alkyl, lower alkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which may be optionally substituted. Preferred aryl groups include phenyl, halophenyl, loweralkylphenyl, napthyl, biphenyl, phenanthrenyl and naphthacenyl.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein may be replaced by one or more of the indicated substituents if such replacement(s) would result in a stable compound. Nitrogen atoms in a ring structure may be quaternized, but such compounds are specifically indicated or are included within the term “a pharmaceutically acceptable salt” for a particular compound. When the total number of O and S atoms in a single heterocyclic ring is greater than 1, it is preferred that such atoms not be adjacent to one another. Preferably, there are no more than one O or S ring atoms in the same ring of a given heterocyclic ring structure.

Examples of monocyclic and bicyclic heterocyclic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl1~1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazmyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocyclic ring structures.

Halo or halogen: refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

Figures:

FIG. 1: Summary of Compounds of Interest: Provides chemical structure for III, VIIₑ, VIIₙ, VIIₜ, VIIₒ, VII_{c}, VIIₐ.

We claim the following:

1. Compounds as described below and in each class, R and R′ may constitute alkyl, aryl, hydrogen, halogen, fluoroalkyl, or heterocyclic.

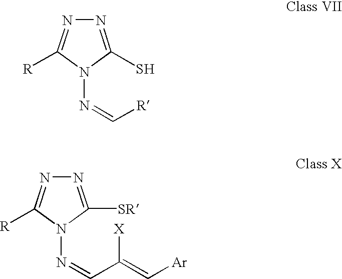

2. A pharmaceutical formulation comprising of a compound of Classes VII and X as defined in claim 1, or an optical isomer or racemate of any chiral analog thereof or a pharmaceutically acceptable salt thereof, optionally in a mixture with a pharmaceutically acceptable diluent or carrier.

3. A method for orally administering said compounds as in claim 1 in a pharmacologically acceptable carrier, said carrier including an ingredient selected from the group consisting of a binding agent, filler, lubricant, disintegrant, wetting agent, inert diluent, surface active agent, dispersing agent, suspending agent, emulsifying agent, edible oil, flavoring agent and mixtures thereof.

4. A method for the topically administering said compounds as in claim 1 in a pharmacologically acceptable carrier in the mouth, said carrier including an ingredient selected from the group consisting of a flavor, sucrose, acacia, tragacanth, gelatin, glycerin and mixtures thereof.

5. A method for nasally administering said compounds as in claim 1 in a pharmacologically acceptable carrier, said carrier including an ingredient selected from the group consisting of a dispersing agent, solubilizing agent, suspending agent and mixtures thereof.

6. A method for administering said compounds as in claim 1 in a pharmacologically acceptable carrier by inhalation, said carrier including a propellant.

7. A method wherein said propellant as in claim 1 is selected from the group consisting of dichlorodifluoromethane, trichlorofluoromethane, dichlorotetrafluoroethane, carbon dioxide and mixtures thereof.

8. A method for administering said compounds as in claim 1 in a pharmacologically acceptable carrier by inhalation or insufflation, said carrier including an ingredient selected from the group consisting of lactose, starch and mixtures thereof.

9. A method for administering said compounds as in claim 1 in a pharmacologically acceptable carrier parenterally, said carrier including an ingredient selected form the group consisting of an anti-oxidant, buffer, bacteriostat, suspending agent, thickening agent, saline, water and mixtures thereof.

10. A method of administering said compounds as in claim 1 in a pharmacologically acceptable carrier rectally, said carrier including an ingredient selected from the group consisting of cocoa butter, polyethylene glycol and mixtures thereof.

11. A method wherein said compounds as in claim 1 to be administered rectally includes an ingredient selected from the group consisting of an antimicrobial agent, an immunosuppressant, a preservative and mixtures thereof.
